(19) United States
(12) Patent Application Publication
(10) Pub. No.: US 2009/0048504 A1
(43) Pub. Date: Feb. 19, 2009

(54) Medical electrode systems and methods

(75) Inventors: Rafael V. Andino, Lawrenceville, GA (US); Christopher Brooks, Glen Head, NY (US); Michael Keating, Hardwick, NJ (US); Courtney Morgan, Alpharetta, GA (US); Donald Van Royen, New York, NY (US)

Correspondence Address: ROPES & GRAY LLP, PATENT DOCKETING 39/361, 1211 AVENUE OF THE AMERICAS, NEW YORK, NY, 10036-8704, US

(73) Assignee: Biofisica Inc., Duluth, GA (US)

(21) Appl. No.: 11/893,815

(22) Filed: Aug. 17, 2007

(51) Int. Cl. A61B 5/04

(52) U.S. Cl. 600/393

(57) ABSTRACT

Systems, methods, and devices are provided for creating and applying electrodes and electrode systems to a wound or skin. An electrode may be applied as a single electrode strip using a dispenser. An electrode may also be cut to size from a single sheet of electrode. Additionally, an electrode may be formed from a plurality of electrode segments which may be connected together. Electrodes can also be incorporated as part of drainage tubes. A medical electrode kit may be provided that includes multiple electrode segments and connectors, multiple electrodes, and multiple control modules. In addition, electrodes or electrode systems may be provided with a color scoring chart. An electrode system may be configured to interface with a selected body part. Electrode systems may include sensors and electrodes configured for application to areas outside of a wound or the skin intended to be treated.

The present invention relates generally to systems and methods for creating and applying medical electrodes and electrode systems to the body for treating wounds and skin with electrical stimulation and corresponding medical kits.

### BACKGROUND OF THE INVENTION

Medical electrodes have been around for some time. More recently, medical electrodes have been used to treat wounds. For example, patients that suffer from conditions which limit the flow of blood to a wound site are often not able to exhibit a normal wound healing process. Factors that can negatively affect the normal wound healing process include diabetes, impaired circulation, infection, malnutrition, medication, and reduced mobility. Other factors such as traumatic injuries and burns can also impair the natural wound healing process.

Active approaches have been employed to decrease the healing time and increase the healing rates of some wounds and ulcers. It has also been shown that specific types of electrical stimulation will alter the wound environment in a positive way so that the normal wound healing process can occur or in some cases occur in an accelerated fashion.

U.S. Pat. No. 6,631,294 to Andino discloses an electrode system that generates a current flow that envelops and permeates a wound site. The system includes two electrodes, adapted and positioned to cause a current to flow from one electrode through the wound to the other electrode. The system describes preconfigured dressings and electrode systems, in various shapes and sizes. However, wounds can be irregularly shaped and sized, such that a preconfigured system may not optimally treat the wound. Thus there is a need for medical electrodes that are customizable for specific applications.

In view of the foregoing, it is an object of the present invention to provide improved systems and methods for creating and applying medical electrodes to wounds and skin. It is a more particular object of the present invention to provide systems and methods for creating and applying medical electrodes in a desired shape and size depending on the application. It is also an object of the present invention to provide a medical kit that includes components for assembling electrodes and applying electrical stimulation to the electrodes. It is also an object of the present invention to provide a color scoring chart as part of an electrode system to allow a user to compare the color of a wound or skin to the color scoring chart. It is also an object of the present invention to provide improved sensors and sensor configurations as part of an electrode system. It is also an object of the present invention to provide an electrode system where at least two electrodes are configured to be coupled to skin that is not the wound or area that is intended to be treated. It is also an object to provide a control module for an electrode system with improved ports for coupling the control modules to electrodes and sensors. It is also an object of the present invention to provide an electrode as part of a drainage tube.

### SUMMARY OF THE INVENTION

These and other objects of the invention are accomplished in accordance with the principles of the present invention by providing systems and methods for creating and applying electrodes and electrode systems to wounds or skin areas of different shapes and sizes.

In one embodiment of the present invention, an apparatus is provided for dispensing an electrode to a surface. The apparatus includes a holder, a supply of conductive material, and a dispenser. According to one arrangement, the supply of conductive material is coiled around an element in the holder, and threaded through the holder toward a distal end. The distal end includes a dispenser, which dispenses the conductive material. In one approach, a user holds the holder and presses the dispenser against the skin or wound. As the user moves the holder proximally, the conductive material is dispensed from the dispenser and remains adhered to the skin. The conductive material may be adhesive, or it may include a conductive adhesive layer that adheres to the wound site.

In another embodiment of the present invention, electrode segments may be provided that can be attached together to construct a larger electrode having a selected shape. The electrode segments may be provided in various shapes and sizes, including straight and curved segments. In one approach, a health care professional may construct an electrode that is shaped to substantially surround and follow the edge of the wound. Another electrode may be placed on the wound. A control module may be provided that is configured to apply a voltage potential across the electrodes to apply a therapy.

In other embodiments of the present invention, the electrodes may be provided as part of an electrode system in preformed shapes and sizes for particular applications. The electrode system may include two electrodes that are configured to be applied to a selected body part. The shape of the surfaces of the electrodes may be configured to interface with the selected body part such as to envelope or partially surround a particular wound. The electrode system may be flexible, or it may be semi-rigid. The electrode system may also include a control module for applying a voltage potential across the electrodes. In various examples, the electrode system may be preconfigured for application to a patient's heel, ankle, foot, toe, knee, elbow, wrist, hand, or finger.

In another embodiment of the present invention, a supply of electrode material may be provided in the form of a sheet. The sheet may be cut to a selected size and shape. The sheet may include connection nodes that are configured to electrically couple the sheet to a control module. In one suitable approach, a health care professional may cut a first electrode from the sheet such that the first electrode is substantially the same size and shape as a wound. The center of the first electrode may then be cut out so that the electrode surrounds the center or the entire wound when applied to the wound site. One or more center electrodes may also be cut from the sheet. A center electrode may be placed in the center of the wound. Both the first electrode and the center electrode may be connected to a control module or power supply.

In accordance with another embodiment of the present invention, a color scoring chart may be provided as part of an electrode or an electrode system. The color scoring chart may include a range of colors, and may be a redness scoring system. The color scoring chart allows a practitioner to compare the color of the wound to the colors of the scoring system. The colors of the chart may have corresponding numbers.

In another embodiment of the present invention, a medical kit may be provided that includes components for applying an electrode or an electrode system to a wound or other body location. The medical kit may include any of the electrodes and components described herein. The medical kit may include electrode segments of multiple different sizes and shapes and connectors that can be used for coupling the electrode segments together to form larger electrodes. The medical kit may also include electrodes sized for application to skin and/or wounds. The medical kit may also include control modules for applying a voltage potential across two or more electrodes and for receiving signals from one or more sensors. The control modules may include ports that selectively couple to the electrodes and sensors. The apparatus for dispensing electrodes and the electrode sheets that can be cut to selected sizes may also be provided as part of the medical kit. In addition, the medical kit may include a diagnostic device, gauze, a scalpel, scissors, tape, and a wound exudates absorber.

In accordance with other embodiments of the present invention, multiple sensors may be provided as part of an electrode system. The sensors may be configured to take measurement from different locations. For example, the voltage potential can be measured at different locations in the wound. In addition, the temperature and pH can also be measured by these sensors. The measurements can be used by the control module or a health care professional to monitor and adjust the therapy that is being applied.

In accordance with other embodiments of the present invention, one or more electrodes may be applied to areas outside of a wound site or the skin that is intended to be treated. For example, two electrodes may be applied on opposite sides of a wound. By applying a voltage potential across the two electrodes, a current may be caused to flow through the wound. A third (or more) electrode or a sensor may also be applied to the wound.

In accordance with other embodiments of the present invention, an electrode may be incorporated as part of a drainage tube.

### BRIEF DESCRIPTION OF THE DRAWINGS

The foregoing and other objects and advantages of the invention will be appreciated more fully from the following further description thereof, with reference to the accompanying drawings.

FIGS. 1A and 1B are sectional views of an illustrative electrode dispenser in accordance with the present invention.

FIG. 2 is a perspective view of an illustrative electrode dispenser applying an electrode to a wound site in accordance with the present invention.

FIG. 3 is a diagram of an exemplary electrode system kit for use in creating and applying an electrode to a wound or skin in accordance with the present invention.

FIG. 4 depict two electrode components prior to and after assembly into a continuous electrode segment in accordance with the present invention.

FIG. 5 depicts an exemplary electrode system applied to a wound site in accordance with the present invention.

FIGS. 6A and 6B depict an exemplary preformed electrode system dressing for application to a patient's heel in accordance with the present invention.

FIG. 7 depicts an electrode sheet being cut to size for application to a wound site in accordance with the present invention.

FIG. 8 depicts an electrode system including a color scoring chart in accordance with the present invention.

FIG. 9 depicts a medical kit in accordance with the present invention.

FIG. 10 depicts a top view of an illustrative electrode system in accordance with the present invention.

FIG. 11 depicts a side view of an illustrative sensor in accordance with the present invention.

FIG. 12 depicts an exemplary electrode system applied to a wound in accordance with the present invention.

FIG. 13 depicts another medical kit in accordance with the present invention.

FIG. 14 is a sectional view of electrode 464 of FIG. 13 taken along line 14-14.

### DETAILED DESCRIPTION OF THE DRAWINGS

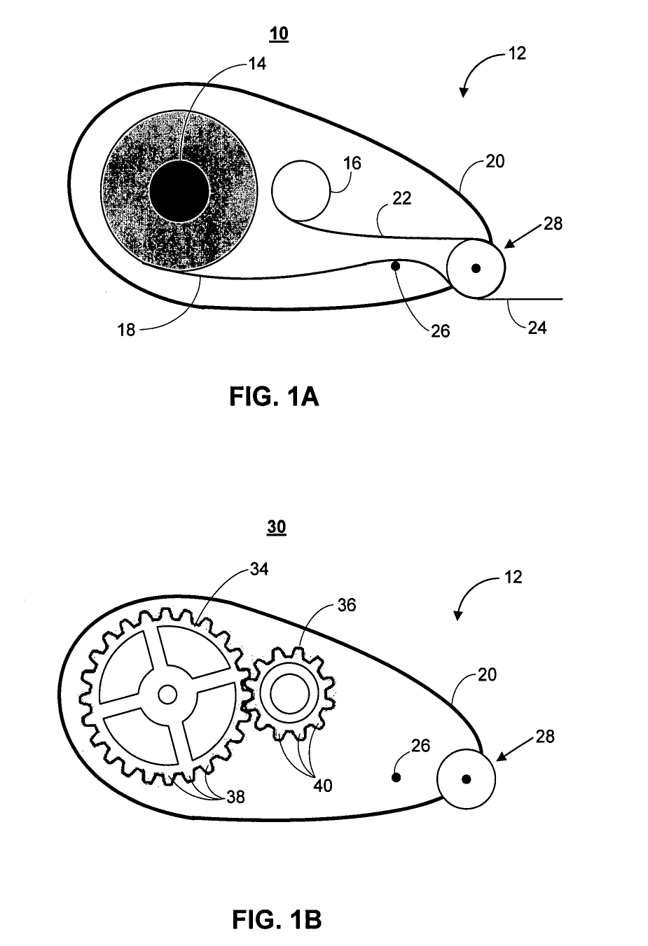

To provide an overall understanding of the invention, certain illustrative embodiments will now be described with reference to FIGS. 1-14. It will be understood by one of ordinary skill in the art that the systems, methods, devices, and medical kits shown and described herein can be adapted and modified for other suitable applications and that such other additions and modifications will not depart from the scope hereof.

In accordance with some embodiments of the present invention, electrodes are provided for application to wounds and skin. The electrodes are capable of being configured into various shapes and sizes. This is particularly beneficial for the application of electrodes to different surface contours and irregular shapes. In one embodiment, the electrodes are used as part of an electrode system configured to apply a therapy to wounds and skin. For example, the electrodes of the present invention may be used with the electrode systems of commonly-assigned U.S. Pat. No. 6,631,294 and commonly-assigned U.S. patent application Ser. No. 11/494,819, filed on Jul. 28, 2006, the contents of both of which are hereby incorporated by reference. The electrode system may include a control module and multiple electrodes.

The electrodes of the present invention may be made of thin metal, metallic paint or pigment deposition, metallic foil, conductive hydrogels, or any other suitable conductive material. In one suitable approach, silver may be used as at least part of the material for the electrodes due to its bactericidal properties. In another suitable approach, conductive hydrogels may be used as the material for the electrodes because of their permeability to oxygen and ability to retain water. Hydrogels are generally clear, viscous gels that protect the wound from desiccating. Both oxygen and a humid environment, for example, are needed for the cells in a wound to be viable. In addition, hydrogels can be easily cast into any shape and size. Various types of conductive hydrogels may be employed, including cellulose, gelatin, polyacrylamide, polymethacrylamide, poly(ethylene-co-vinyl acetate), poly(N-vinyl pyrrolidone), poly(vinyl alcohol), HEMA, HEEMA, HDEEMA, MEMA, MEEMA, MDEEMA, EGDMA, methacrylic acid based materials, and siliconized hydrogels. PVA-based hydrogels are inexpensive and easy to form. The conductivity of such hydrogels can be changed by varying the salt concentration within the hydrogels. By increasing the salt concentration within a hydrogel, the conductivity of the hydrogel increases. In addition, the diffusion properties of the hydrogel can be varied as a means of optimizing the transport and conductivity properties of the hydrogel. Parameters such as pore size, which can be affected by the degree of cross linking, and water content, which can be affected by the addition of ionizing monomeric groups such as methacrylic acid or side groups such as urea or amine groups, can be varied to achieve desired hydrogel properties. Bulk water holding properties of the hydrogel can be changed during the gelation process through, for example, the use of the isocyanate reaction with water to generate carbon dioxide thus forming a hydrogel with an open cell structure providing voids for water (like a sponge). This provides the opportunity to design a desired controlled release of moisture to the wound.

The electrodes may include a nonconductive backing layer that may provide support for the electrodes. In addition, the electrodes themselves may have adhesive properties (e.g., hydrogel) or an electrically conductive adhesive may be applied to the surface of the electrodes for attaching the electrodes to a surface such as a wound or skin.

The control module may be coupled to the electrodes to provide a voltage potential across the electrodes. The control module may be the same or substantially similar to the control modules disclosed in commonly-assigned U.S. Pat. No. 6,631,294 and commonly-assigned U.S. patent application Ser. No. 11/494,819, filed on Jul. 28, 2006, which are incorporated by reference herein. In various arrangements, the control module may include a processor, a display, a memory, a power supply, a timer, and a user input device. A user or health care professional may use the control module to select or alter the therapy applied to the wound via the electrodes. The control modules used in accordance with the present invention may provide a closed loop control system where the skin and wound form an integral part of the circuitry. For example, the control modules may be configured to provide a constant current between or voltage across the two or more electrodes applied to the wound and/or skin. In addition, the control modules may be configured to provide a constant current density across an area or range of areas associated with the wound and/or skin.

FIG. 1A is a sectional view 10 of an illustrative electrode dispenser 12 in accordance with the present invention. Electrode dispenser 12 is configured to apply an electrode to a wound or skin. Electrode dispenser 12 includes a supply element 14, a receiving element 16, an electrode strip 18, and housing 20. Electrode strip 18 includes a backing 22 and an electrode tape 24. As shown in FIG. 1A, electrode strip 18 is a continuous length of electrode and a supply of electrode strip 18 is wrapped around supply element 14. Electrode strip 18 extends from supply element 14, passes by guiding element 26, wraps partially around dispensing element 28, and is received at receiving element 16. During application of electrode tape 24 to a wound or skin, electrode strip 18 moves from supply element 14 to receiving element 16 and electrode tape 24 separates from backing 22 at or near dispensing element 28 for application to the wound or skin. Backing 22 may be a nonstick material to facilitate the separation of electrode tape 24. Backing 22 continues and is received at receiving element 16.

As illustrated, dispensing element 28 is a rotatable cylinder that is capable of rotating about its center. Dispensing element 28 may have raised sides to keep electrode strip 18 from sliding in either axial direction off of the dispensing element. In another suitable arrangement, dispensing element 28 may be a stationary structure that electrode strip 18 slides against. In such an arrangement, dispensing element 28 may be any suitable shape to facilitate electrode tape 24 from separating from backing 22. For example, dispensing element 28 may be pointed to facilitate separating electrode tape 24 from backing 22.

FIG. 1B is another sectional view 30 of electrode dispenser 12 showing first gear 34 and second gear 36. First gear 34 and second gear 36 are attached adjacent to the sides of supply element 14 and receiving element 16, respectively, or are a part of them. First gear 34 includes a first number of gear cogs 38 and the second gear 36 includes a second number of gear cogs 40. The first number of gear cogs 38 interfit with the second plurality of gear cogs 40, such that rotation of one of the gears 34 or 36 causes the opposite rotation of the other. As shown, first gear 34 includes more gear cogs than second gear 36. This is merely illustrative. Gears 34 and 36 may include the same number of gear cogs or second gear 36 may include more gear cogs than first gear 34. When first gear 34 rotates in the counter-clockwise direction, electrode tape 24 is dispensed from electrode dispenser 12 and backing 22 is wound around receiving element 16.

In one suitable configuration, first and second gears 34 and 36 and their respective supply and receiving elements 14 and 16 are sized and shaped such that the length of electrode strip 18 released from supply element 14 is substantially equal to the length of backing 22 wound around receiving element 16. In another suitable configuration, first and second gears 34 and 36 and their respective supply and receiving elements 14 and 16 are sized and shaped such that the length of electrode strip 18 released from supply element 14 is less than the length of backing 22 wound around receiving element 16. In such a configuration, electrode strip 18 will be caused to undergo tension between supply element 14 and receiving element 16 as electrode tape 24 is being applied. In order to account for the different lengths being supplied and received on supply element 14 and receiving element 16, backing 22 may be made of a flexible material. In another suitable approach, backing 22 may be frictionally coupled to receiving element 16 such that the backing is capable of moving relative to receiving element 16 under a certain amount of force. In another suitable approach, receiving element 16 may be frictionally coupled to second gear 36 such that receiving element 16 is capable of moving relative to second gear 36 under a certain amount of force.

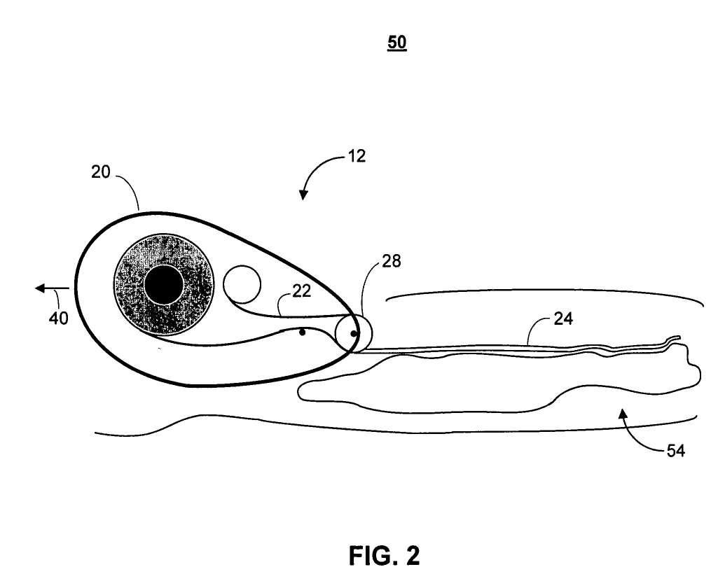

FIG. 2 is a perspective view 50 of an electrode dispenser 12 in use in accordance with the present invention. Housing 20 of electrode system 12 may be made in a shape and size suitable for holding by an operator. An operator can apply electrode tape 24 from electrode dispenser 12 by holding housing 20 and pressing dispensing element 28 against a surface, such as the skin of a patient, and moving electrode dispenser 12 in direction 40. Electrode tape 24 disengages from backing 22 at or near dispensing element 28, remaining affixed to the selected surface. The side of electrode tape 24 that is applied to the surface may be may be adhesive, or it may include an adhesive component as discussed above. Backing 22 continues on dispensing element 28, returns into housing 20, and is accumulated about receiving element 16.

According to one example, a health care professional may apply electrode tape 24 on skin around a wound, to substantially surround a wound. Electrode tape 24 may be flexible such that the health care professional may easily apply the tape in a desired shape. For example, this may be achieved by moving dispensing element 28 of electrode dispenser 12 in the desired shape across the receiving surface.

As described above, supply and receiving elements 14 and 16 are manually operated when the dispensing element 28 of electrode dispenser 12 is manually moved across a surface. In another suitable arrangement, a motor powered by, for example, a battery may be included in housing 20 to automatically advance electrode strip 18. In this arrangement a button may be provided on housing 20 that will turn the motor on and off. This may be useful if the element is being applied to a sensitive wound or skin.

In an alternative embodiment, dispensing element 28 of electrode dispenser 12 may not be included and receiving element 16 may act as both the receiving element and the dispensing element.

Once electrode tape 24 has been applied to a surface, the tape may be cut to separate the applied electrode tape from electrode dispenser 12. Electrode dispenser 12 may be used multiple times to apply electrodes to a surface. The applied electrode tape can then be coupled to a control module using the techniques described further below, including having connection nodes on the electrode tape.

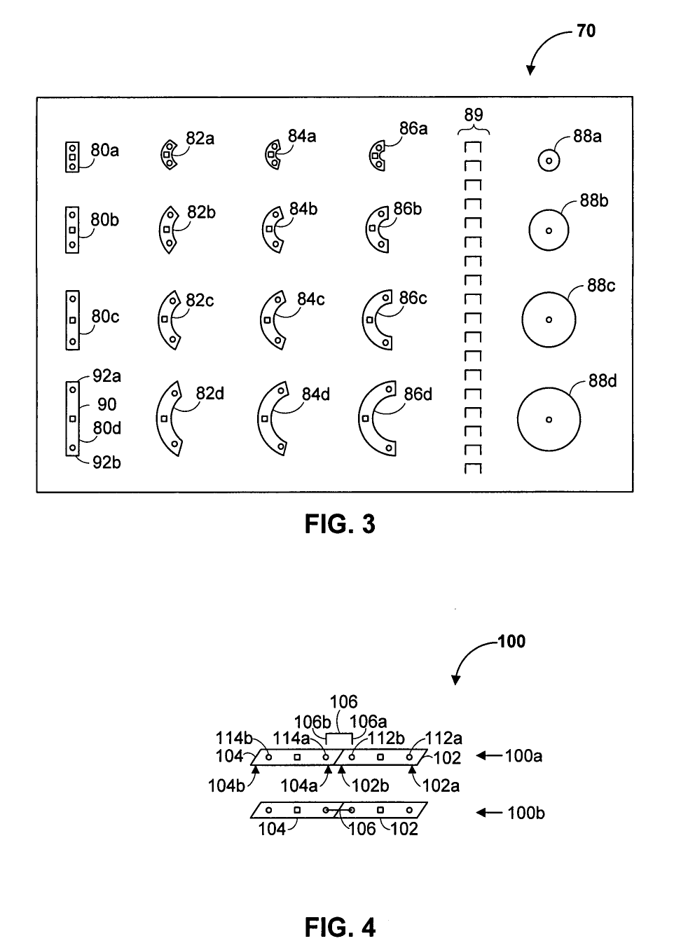

In another embodiment of the present invention, an electrode can be assembled from multiple electrode components to create an electrode of a desired size and shape. FIG. 3 is a diagram of an exemplary electrode system kit 70 for use in creating and applying an electrode to a wound or skin. Electrode system kit 70 includes electrode components 80a-80d, 82a-82d, 84a-84d, 86a-86d, 88a-88d and connectors 89. Electrode system kit 70 may include several ones of each of electrode components 80a-88d. Electrode components 80a-88d may include a nonconductive backing layer and an electrically conductive layer. Electrode components 80a-88d may each include a connection node, such as node 90 of electrode component 80d, which may be used to connect the respective electrode component to a control module or power supply. The nodes may extend from the electrically conductive layer through the nonconductive layer. Electrode components 80a-86d may also include first and second attachment receptacles, such as receptacles 92a and 92b of electrode component 80d, located at the distal ends of the respective electrode components. The attachment receptacles may be used to electrically and/or physically attach respective electrode components together using connectors 89. In another suitable arrangement, electrode components 80a-86d may not include attachment receptacles, and may be attached using alternative attachment mechanisms. For example, the ends of the electrode components may be overlapped when applied to a surface, thereby making an electrical connection between the components. The electrode components 80a-86d are preferably assembled together to create a longer continuous electrode segment having a selected size and shape, as will described in greater detail below with respect to FIG. 4.

The electrode components may be provided in straight and curved pieces. The curved pieces may be any suitably sized arcs having any selected radius. According to one approach, the curved electrode segments are arcs having angles of 180 degrees or less, having any suitable radius of curvature, including, for example, about 0.5 cm, about 1 cm, about 2 cm, about 4 cm, about 6 cm, about 8 cm, about 10 cm, about 15 cm, about 20 cm, about 30 cm, about 50 cm, about 100 cm, or more than 100 cm. The straight and curved electrode segments may have any suitable length, including, for example, about 0.5 cm, about 1 cm, about 2 cm, about 3 cm, about 4 cm, about 5 cm, or more than 5 cm.

FIG. 4 depicts two electrode components prior to and after assembly into a continuous electrode segment in accordance with the present invention. The top part of FIG. 4 shows electrode segments 100a including electrode components 102 and 104, and a connector 106. Electrode component 102 includes first and second receptacles 112a and 112b and electrode component 104 includes first and second receptacles 114a and 114b. First receptacles 112a and 114 a are located on first ends 102a and 104a of electrode components 102 and 104, respectively while second receptacles 112b and 114b are located on second ends 102b and 104b of electrode components 102 and 104, respectively. According to the illustrative arrangement, first end 104a of electrode component 104 is aligned with second end 102b of electrode component 102.

Connector 106 includes protrusions 106a and 106b, and is sized and shaped for interfitting with adjacent receptacles such as receptacles 112b and 114a. As shown in FIG. 4, protrusion 106a can be inserted into receptacle 112b, and protrusion 106b can be inserted into aperture 114a, thereby electrically and physically connecting electrode components 102 and 104, as shown in electrode segment 100b. Additional connectors 106 may be used to attach together additional electrode components, forming a longer electrode segment.

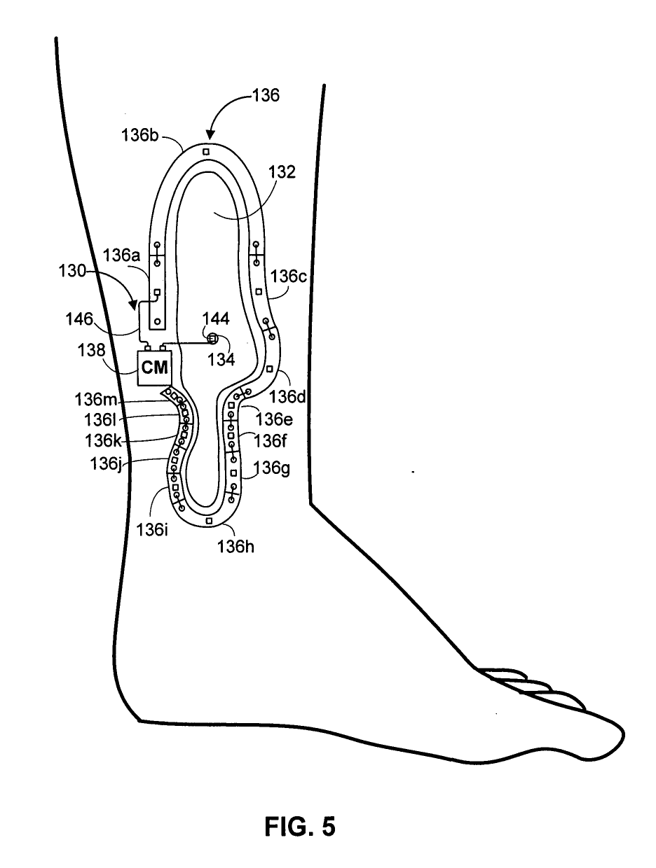

FIG. 5 depicts an exemplary electrode system 130 applied to a wound 132 in accordance with the present invention. The electrode system 130 includes a center electrode 134, an external electrode 136, and a control module 138. External electrode 136 is assembled from a plurality of electrode components 136a-136m. Center electrode 134 and external electrode 136 of electrode system 130 may be assembled from a kit, such as electrode system kit 70 of FIG. 3, and the electrode components 136a-136m may be substantially the same as the electrode components 80a-86d of FIG. 3. In one suitable approach, electrode components 136a-136m may be selected by a health care professional such that the assembled external electrode 136 is sized and shaped to partially or completely surround wound 132. Electrode components 136a-136m may be attached using any suitable method, such as using connectors 89 and 106 of FIGS. 3 and 4.

Center electrode 134 and the surrounding electrode 136 may be connected to the control module 138 via conductive cables 144 and 146. Conductive cables 144 and 146 are connected to the control module and to connector nodes in electrodes 134 and 136. According to one approach, only one electrode component 136a of external electrode 136 is connected to control module 138, and the electrode components 136a-136m are sufficiently attached such that any voltage applied by the control module 138 to external electrode component 136a is substantially equally applied to the entire external electrode 136. In another suitable approach, control module 138 may be connected to two or more electrode components to ensure that the voltage is substantially the same along the entire length of external electrode 136. According to another suitable approach, the electrode components of external electrode 136 may not include connection nodes, and an external electrode attachment device may be used to connect electrode 136 with control module 138.

The foregoing is merely illustrative. The electrode components may be assembled in any suitable size or shape depending on the desired application.

In accordance with another embodiment of the present invention, the dressing may be sprayed or painted onto the wound. The spray dressing may comprise a conductive material which may be in liquid or atomized form that cures on contact or is cured by exposure to an agent or UV energy source. An example of a material that cures on contact is polysaccharide alginate which crosslinks in the presence of calcium ions (Ca can be supplemented if not enough is present in the tissue). An example of a material cured in place by the addition of an agent is PVA (polyvinyl alcohol) upon addition of borate ions. An example of a suitable UV cured material is PVA that has been chemically modified to contain photo initiated cross linking side groups.

The spray dressing may cover the anode and cathode leads or alternatively, the anode and cathode leads may be attached or applied to the spray dressing after the dressing has been cured. According to one example, a sprayed or painted dressing may be particularly useful on burn wounds. Burn wounds are very delicate and can be extremely painful. By using such a sprayed on or painted dressing, pain and tissue damage can be minimized compared with adhesive based tape type dressings.

Removal of hydrogels formed as described above in the alginate example can be facilitated by a wash with a chelating agent such as EDTA (ethylenediamine tetraacetic) or DPTA (diethylenetriaminepentaacetic acid) to remove the Ca and break the cross link.

According to another arrangement, the electrodes may be configured to provide a voltage gradient across the wound without a control module such as control module 138 shown in FIG. 5. For example, dissimilar metals such as Co—Cr alloy and Ti may be integrated into the electrodes such that the body's own fluids and/or tissue may be the catalyst to drive a potential gradient through the conduction mediated by the characteristics of the electrodes. The electrodes can be configured in any of a variety of geometries such as sheets, perforated foils, screens or meshes or fine wires formed in specific shapes maintaining the consideration of diffusion such that the relative placement of the anode(s) and cathode(s) imparts a degree of control on the potential gradient and therefore the current flow patterns. Additionally, the electrode may be made from hydrogel or include a hydrogel layer, such that the hydrogel includes specific metals, chemicals, or compounds which react to produce a voltage gradient across the wound. In another suitable approach, other encapsulated reagents may be used as a catalyst to create a voltage gradient across the wound. In other suitable approaches, gels, pastes, or other agents may be applied to the wound to create a voltage gradient across the wound. These gels, pastes, or other agents may contain dissimilar metals which can induce the same type of galvanic current. Additionally, these gels, pastes, or other agents may be oppositely charged of sufficient differential voltage potential and charge densities to cause a current to flow as described in commonly-assigned U.S. Pat. No. 6,631,294. The foregoing may be accomplished by using multiple layers in the electrodes. For example, an electrode may comprise a first layer that includes a first metal, a second layer that does not include any metal, and a third layer that includes a second dissimilar metal. The second layer may have a high resistance and the first and third layers may have low resistances.

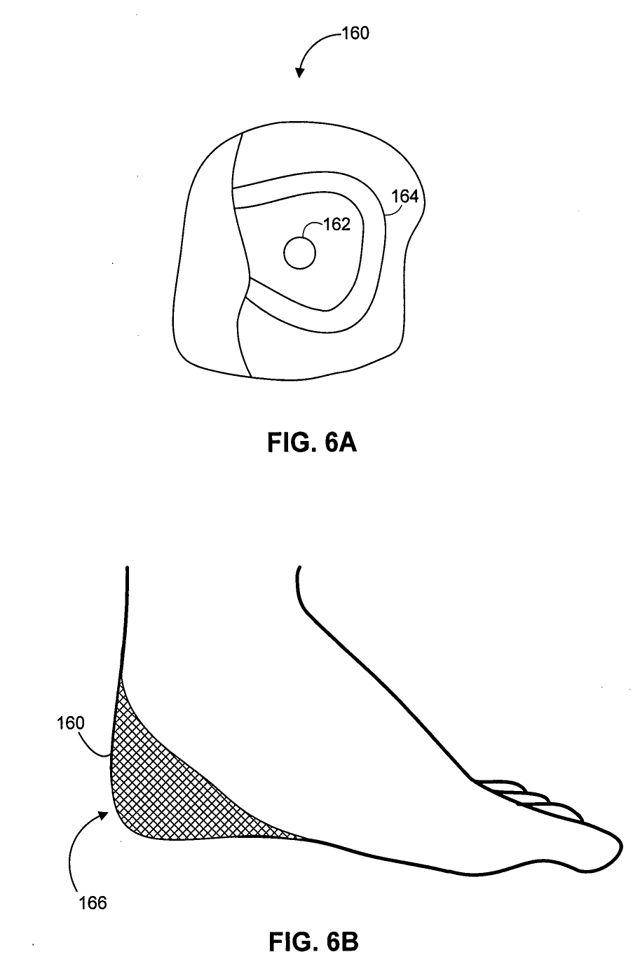

In accordance with another embodiment of the present invention, the electrodes may be provided in preformed shapes and sizes for particular applications. The shape of the surfaces of the electrodes may be configured to interface with the selected body part. FIGS. 6A and 6B depict an exemplary preformed electrode system dressing 160 for application to a patient's heel 166. The heel is a location where skin ulcers may form and where it may not be an easy to apply electrodes due to its shape. Electrode system dressing 160 may include a center electrode 162, an external electrode 164, and a control module for applying a voltage potential across the electrodes and thus apply a therapy to a wound. As shown, external electrode 164 may be positioned substantially around the edge of dressing 160, and center electrode 162 may be positioned substantially in the center of dressing 160. Dressing 160 may be flexible, or it may be semi-rigid. In another suitable approach, the external electrode may be a continuous sheet of electrode, substantially the same size as dressing 160, and the center electrode may be a separate smaller electrode which is placed in the center of the wound. A gap or an insulation layer may separate the center electrode from the continuous sheet. Additionally, the center electrode 162 can be decoupled from the outer electrode 164 such that electrode 162 lies in a plane that is outside of electrode 164. This allows for electrode 162 to lie in direct apposition of a deep wound (within the wound cavity) where outer electrode 164 can lie outside of that deep wound.

The preformed electrode system dressing shown in FIGS. 6A and 6B is merely illustrative. The preformed electrode system may be preconfigured for application to any selected body part, including, for example, an ankle, a foot (e.g., for the plantar and dorsal surfaces), a toe, a knee, an elbow, a wrist, a hand, or a finger, or the stump remaining from an amputated member of the body such as a leg or arm or a portion thereof. The preformed electrode may be useful for any location on the body where other standard sized electrodes would be difficult to apply. The preformed electrode system dressings may be provided in multiple sizes to fit patients of different sizes.

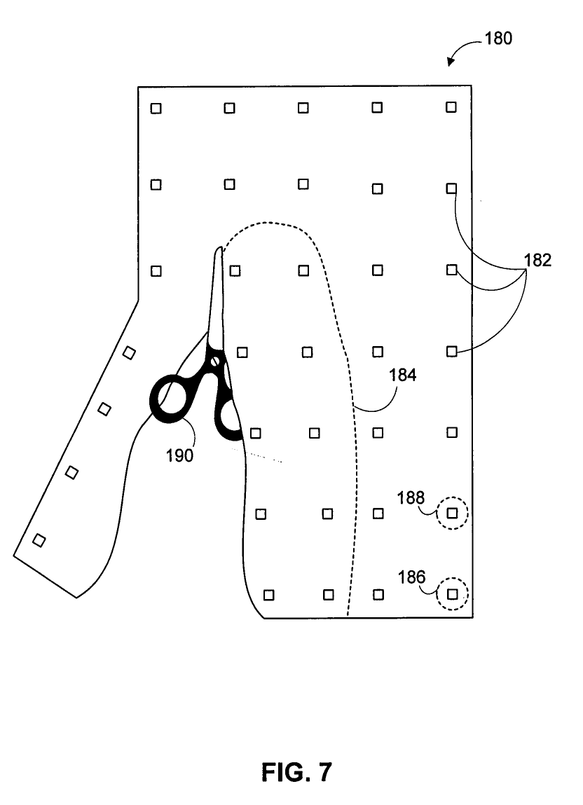

In accordance with another embodiment of the present invention, the electrodes may be cut from an electrode sheet into any suitable sizes or shapes. FIG. 7 depicts an electrode sheet 180 being cut to size for application to a wound site in accordance with the present invention. Electrode sheet 180 may be a solid piece of electrode that includes one or more layers. For example, electrode sheet 180 may include a nonconductive backing layer and a conductive layer. The conductive layer may have adhesive properties or an adhesive layer may be applied to the conductive layer for attaching the conductive layer to a surface. Electrode sheet 180 may also include a removable protective layer that covers and protects the surface of the electrode sheet that is to be applied to a surface. The removable protective layer can be removed prior to application. Electrode sheet 180 may include a plurality of connector nodes 182 that may be similar to the connector nodes described above in connection with FIGS. 3-5. Connector nodes 182 may be used to connect the electrode sheet 180 to a control module or power supply. Electrode sheet 180 may be cut by a health care professional to form an electrode of any suitable size or shape. As shown, electrode sheet 180 is being cut with scissors 190. This is merely illustrative. Electrode sheet 180 may be cut using any suitable means including, for example, a knife or sheers. If electrode sheet 180 continues to be cut along the dotted line 184, it will form a shape suitable for application to wound 132 of FIG. 5. The center portion of the electrode may also be cut out so that the electrode is similar in shape to external electrode 136 of FIG. 5. Similarly, center electrodes 186 or 188 may be cut from the same sheet 180. In a preferred approach, an electrode cut from the sheet 180 includes one or more connector nodes 182. Sections of electrode sheet 180 may be cut out for use, leaving the remaining sections for future use.

Electrode sheet 180 may be provided in any selected shape or size. Electrode sheet 180 may, for example, be rectangular with a width of about 2, 5, 10, 20, 30, 50 or more centimeters and a length of about 2, 5, 10, 20, 30, 50, 100 or more centimeters. The width and/or length of the sheet 180 may also be less than about 2 cm. In one suitable approach, electrode sheet 180 may be rolled up for storage. In another suitable approach, electrode sheet 180 may be supplied as a package of multiple sheets.

Connection nodes 182 of electrode sheet 180 may be spaced at any suitable distance on sheet 180. For example, along the width of electrode sheet 180, connection nodes 182 may be positioned about 1, 2, 3, 4, 5, 7, 10, or more centimeters apart, and along the length of electrode sheet 180, connection nodes 182 may be positioned about 1, 2, 3, 4, 5, 7, 10 or more centimeters apart.

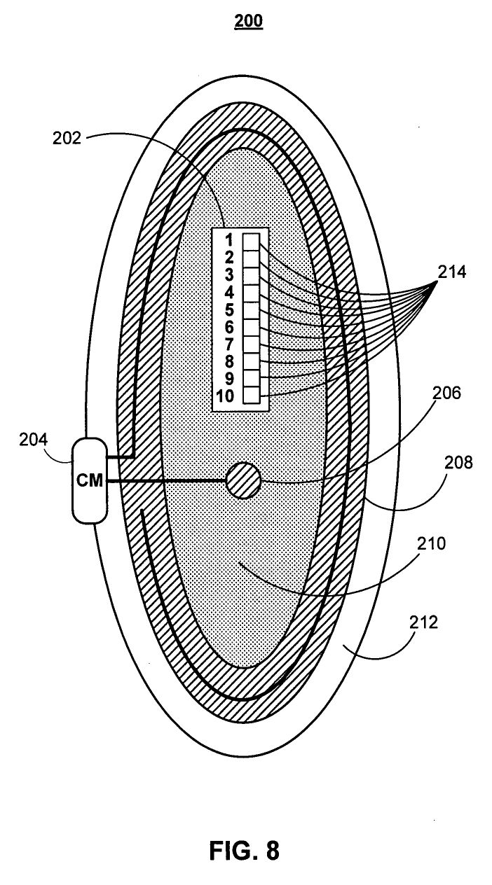

In accordance with another embodiment of the present invention, the electrodes may be provided with a means for determining a treatment status for the wound or skin to which they are attached. FIG. 8 depicts an electrode system 200 including a color scoring chart 202 having multiple colors 214 in accordance with the present invention. Electrode system 200 also includes a control module 204, a center electrode 206, an external electrode 208, a transparent or semitransparent electrically insulative layer 210, and a top overlay layer 212. Color scoring chart 202 may be positioned on top of the electrically insulative layer 210, allowing a user to directly compare colors 214 of chart 202 with the wound color. Color scoring chart 202 may be a redness scoring system. Color scoring chart 202 as shown includes ten colors, and each includes a corresponding number. The colors may be various shades of red, ranging from a deep red to a light pink. For example, the color ‘10’ may be a deep red, the color ‘1’ may be a light pink, and each of the intermediate colors from ten to one may be a shade lighter than the previous color. The color of a wound can indicate its condition, and a health care professional may use the color scoring chart 202 to monitor the color and thus condition of a wound. Alternatively, the colors used may include white or green (or a combination thereof) to indicate the presence of an infection or a colonization of the wound bed. Black can also be used to indicate the presence of necrotic tissue.

The color scoring chart shown in FIG. 8 is merely illustrative. The color scoring chart may include any suitable number and types of colors. For example, the color scoring chart may include between about 3 colors and about 10 colors. However, the color scoring chart may include less than 3 colors or more than 10 colors. The location of the color scoring chart on electrode system 200 is merely exemplary. The color scoring chart may be located on any suitable component of the electrode system, including the control module, the center electrode, and the external electrode. In another alternative, insulative layer 210 may not be transparent, but a portion of the layer can be configured to peel back to reveal the wound for comparison with the color scoring chart. This portion of insulative layer 210 may be returned after the comparison.

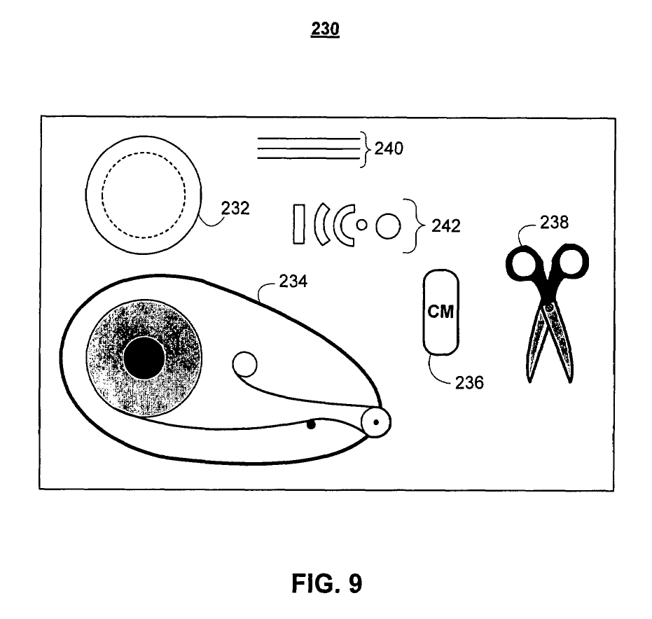

In accordance with another embodiment of the present invention, a medical kit may be provided that includes components for applying an electrode or an electrode system to a wound site. FIG. 9 depicts an illustrative medical kit 230 in accordance with the present invention. Medical kit 230 may include a dressing 232, an electrode dispenser 234, a control module 236, scissors 238, cables 240, electrode components 242, and/or the electrodes depicted in system 70 and sheet 180. Dressing 232 may be any suitable wound dressing, for example, a gauze dressing, a transparent adhesive dressing, an absorption dressing, or a semipermeable polyurethane foam dressing. Medical kit 230 may include multiple dressings having various shapes and sizes. Electrode dispenser 234 may be similar to electrode dispense 12 shown in FIGS. 1A, 1B, and 2. Medical kit 230 may include multiple electrode dispensers 234 of different lengths, widths, and types.

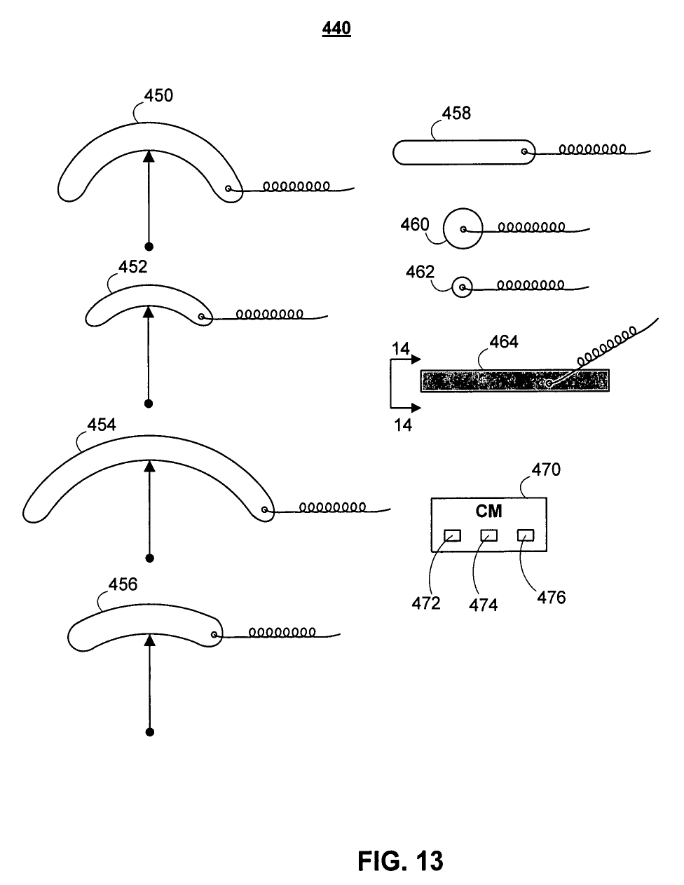

Electrode components 242 may be similar to electrode components shown in FIG. 3 and in FIG. 13 discussed further below. Control module 236 may be included in the medical kit to apply a voltage potential across two or more electrodes. Control module 236 may be connected to the electrodes using cables 240. Cables 240 may be any suitable electrically conductive connections for coupling control module 236 to the electrodes. Cables 240 may be supplied in medical kit 230 in different lengths.

Medical kit 230 may include any of the electrodes and components described herein. Medical kit 230 may also include instructions and advice for creating and applying electrodes and applying therapies to the wound and skin. In addition, medical kit 230 may include tools useful for treatment preparation or wound debriedment such as gauze, scalpels, tape, wound exudates absorbers such as alginates, and gauze or wound odor absorbers such as charcoal.

Medical kit 230 may also include a diagnostic device. The diagnostic device may be a multi-meter to measure the current and/or voltage or other biosensors to measure, for example, the specific biochemistry of the wound. The kit may also include any items commonly found in first-aid kits, such as surgical tape, alcohol swabs, latex gloves, and bandages.

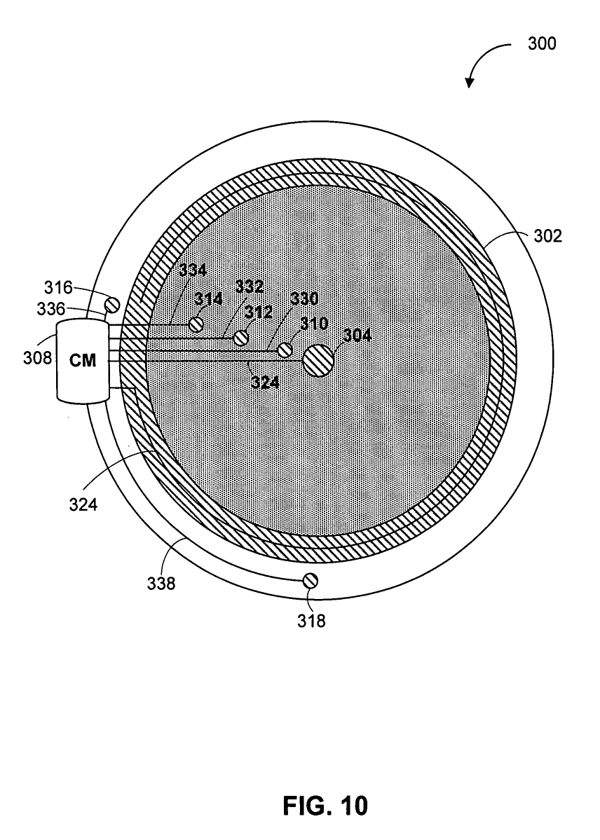

In accordance with other embodiments of the present invention, sensors may be provided and added to the wound and/or skin surrounding the wound. FIG. 10 is a top view of an illustrative electrode system 300, including electrodes 302 and 304, and control module 308 in accordance with the present invention. According to the illustrative arrangement, electrode system 300 includes feedback sensors 310, 312, 314, 316, and 318. Conductive leads 322 and 324 connect electrodes 302 and 304 to control module 308. Additionally, leads 330, 332, 334, 336, and 338 connect feedback sensors 310, 312, 314, 316, and 318 to control module 308.

According to the illustrative arrangement of FIG. 10, control module 308 is coupled to feedback sensors 310, 312, 314, 316, and 318. Each of feedback sensors 310, 312, 314, 316, and 318 may be configured to detect one or more factors that affect wound growth or other treatment factors, and to provide an output to control module 308. Feedback sensors 310, 312, 314, 316, and 318 may be configured to measure the voltage potential across various locations in the wound. For example, feedback sensor 316 may be a reference sensor, located on healthy skin, and the voltage potential may be measured between feedback sensor 316 and feedback sensors 310, 312, and 314, thus providing the voltage potential at various distances from the center of the wound. In another example, sensors, such as feedback sensor 318, may be placed around the wound.

In various examples, measurements from feedback sensors 310, 312, 314, 316, and 318 may be taken while a therapy is being applied or when therapy is not being applied. Measurements taken at different points in time may be compared. For example, sensor measurements may taken while therapy is being applied, and at selected time intervals thereafter. Voltage measurements may be monitored to determine how quickly the voltages change at various locations in the wound, at what level the voltages stabilize, and the length of time it takes for the measurements to stabilize. These measurements may be used by the control module or a medical professional to determine the course of therapy to apply to the wound (e.g., the voltage strength, and the time interval between applications). In one example, the sensor measurements are taken continuously.

Feedback sensors 310, 312, 314, 316, and 318 are shown as individual standalone sensors. In another arrangement, one or more of feedback sensors 310, 312, 314, 316, and 318 may be incorporated into electrodes 302 or 304 or other components of the wound dressing.

In other arrangements, feedback sensors 310, 312, 314, 316, and 318 may be any suitable type of sensor, including, for example, a reactive sensor, an electrochemical sensor, a biosensor, a biochemical sensor, a physical property sensor, a temperature sensor, a sorption sensor, a pH sensor, a voltage sensor, a current sensor, and any suitable combination thereof. Feedback sensors 310, 312, 314, 316, and 318 may be configured to detect any suitable factor or factors that affect the treatment of skin or wound growth, including, for example, the natural current of injury of the wound, the amount of peroxide being generated by an electrode placed in the wound or the amount of peroxide present in the wound bed, the temperature of the wound, and the temperature of the skin surrounding the wound. Feedback sensors 310, 312, 314, 316, and 318 may be configured to detect other treatment factors including chemical levels, the amount of oxygen, the amount of carbon dioxide, pH, fibrium, albumin, sodium salts, up regulation or down regulation of genes, calcium, red blood cells, white blood cells, bacterial fauna, ions, and cations in the wound. Feedback sensors 310, 312, 314, 316, and 318 may be placed in any suitable location on the patient, including on the treated part of the skin, in the center of a wound, on an edge of the wound, or on healthy skin surrounding the wound.

In addition, feedback sensors 310, 312, 314, 316, and 318 may be configured to examine the surface of the electrodes to observe changes over time to determine the chemistry of what is occurring in the wound bed. Feedback sensors 310, 312, 314, 316, and 318 may be configured to detect the liberation of selected growth factors by the wound or surrounding tissue, the liberation of selected ionic species by the wound or surrounding tissue, or the liberation of selected biological chemicals or compounds that relate to the wound or surrounding tissue such as genes.

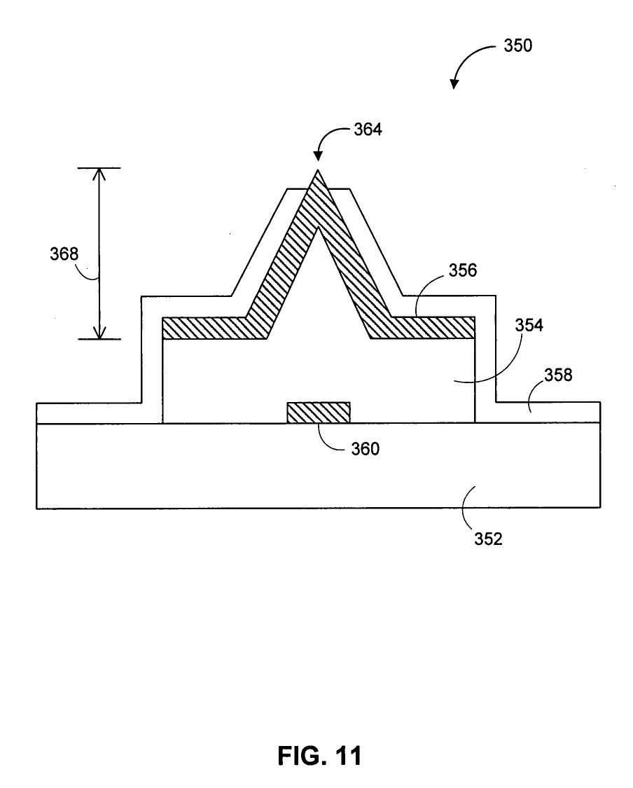

An illustrative feedback sensor that may be used in accordance with the present invention is shown in FIG. 11. FIG. 11 depicts a sectional view of a sensor 350, including a substrate 352, a needle 354, a conductive coating 356, an insulative coating 358, and a conductive lead 360. Needle 354 may be constructed of a conductive material such as silicon that is anodically bonded to substrate 352. Substrate 352 may, for example, be a glass substrate. Needle 354 may be micromachined in any suitable height. In some embodiments, the height may be less than about 500 μm. The shank of needle 354 has a height 368, which may, for example, be less than about 200 μm. Conductive lead 360 is electrically connected to the base of silicon needle 354, and may extend to an edge of the substrate 352. Conductive lead 360 may be constructed of any suitable conductive material such as platinum or platinum silicide. Conductive coating 356 covers tip 364 and the shank of needle 354, and may be, for example, be metal such as platinum, silver, or silver chloride. Insulative coating 358 covers substrate 352, and the base and shank of needle 354, leaving tip 364 of the needle 354 exposed. Insulative coating 358 may be made of any suitable insulative material such as silicon nitride.

Tip 364 of needle 354 is designed to pierce a top layer of skin or a top layer of a wound. During use on skin, needle tip 364 may be positioned, for example, between about 50 μm and about 200 μm beneath the skin surface, and it may be positioned between about 100 μm and about 150 μm beneath the skin surface. In one example, needle 354 pierces the high resistance stratum corneum of the skin, but not the basement membrane. Needle tip 364 is sharp, and may have a radius of less than 10 μm. In one arrangement, sensor 350 measures voltage potential.

Sensor 350 shown in FIG. 11 includes only one needle 354. According to other arrangements, sensor 350 may include multiple needles 354, and it may include an array of needles 354. While the sensor 350 configured to measure voltage potential, in other embodiments, the sensor 350 may be used to apply a voltage to a wound.

In accordance with other embodiments of the present invention, one or more electrodes may be applied to areas outside of the wound site or area to be treated. For example, two (or more) electrodes may be provided on healthy skin on opposite sides of a wound. By applying a voltage potential across the two electrodes, a current may be caused to flow through the wound site providing a similar beneficial effect as in embodiments where at least one electrode is located in the wound site. One advantage of using electrodes on healthy skin is with regard to sterilization. An electrode placed on healthy skin may not need to be sterilized or may be sterilized to a standard that is lower than the standard for electrodes intended for use in the wound site.

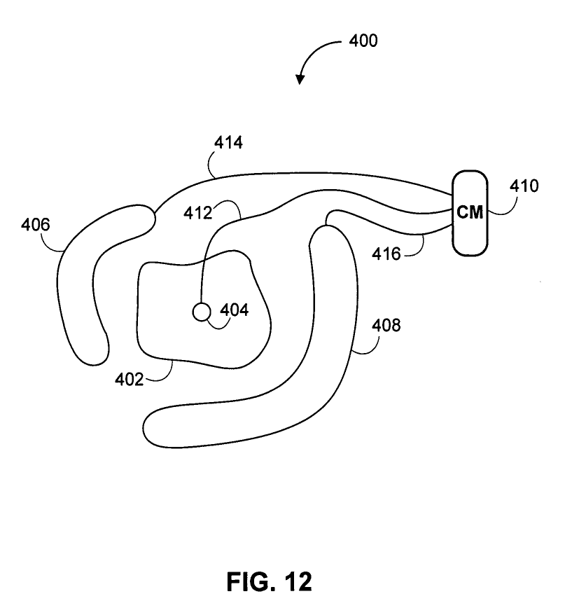

FIG. 12 depicts an illustrative electrode system 400 applied to a wound in accordance with these further embodiments of the present invention. Electrode system 400 includes electrodes 404, 406, and 408. Electrodes 406 and 408 are located outside of wound site 402. Electrode 404 is located within wound site 402. Conductive leads 412, 414, and 416 couple electrodes 404, 406, and 408 to control module 410.

Outside electrodes 406 and 408 may be of any suitable size or shape. As shown in FIG. 12, electrodes 406 and 408 are in the shape of arcs of different radii and different lengths. Electrodes 406 and 408 may be formed, selected or assembled in accordance with the foregoing embodiments. In addition, electrodes 406 and 408 may be selected from electrodes 450, 452, 454, 456, 458, 460, and 462 shown in FIG. 13. For example, electrodes 452, 454, and 462 of FIG. 13 may be used as electrodes 406, 408, and 404, respectively, of FIG. 12. The size and shape of electrodes 406 and 408 may be selected to partially surround different areas of the wound 402. In one suitable approach one of the electrodes may be selected to substantially surround the wound and the other electrode may be selected to partially surround a different area of the wound. In another suitable approach, the two electrodes may be selected to approximately equally surround the wound. The spacing between the ends of the two surrounding electrodes may be large, small, or none. While the foregoing describes there being two outside electrodes, any suitable number of outside electrodes may be used such as 3, 4, 5 or more.

Electrode system 400 may apply any suitable therapy as described herein. For example, electrode 404 may be used as a cathode in combination with electrodes 406 and 408, which may be used as anodes. The therapy may vary such that electrodes 406 and 408 may alternatively be activated to vary the therapy across the wound. In another suitable approach voltage potential may be applied across electrodes 406 and 408. Electrode 404 may be replaced with a sensor to provide feedback to the control module or may not be included at all. For example, the sensor may be configured to detect electrical, chemical, and biological factors described herein. The control module may respond to the sensor measurements to vary the therapy such as by varying the voltage, current, current density, polarity of the electrodes, or any other suitable aspect of the therapy.

FIG. 13 depicts another illustrative medical kit 440 in accordance with the present invention. Illustrative medical kit 440 may be used, for example, to set up and apply an electrode system, such as electrode system 400 shown in FIG. 12, to a wound site. Electrodes 450, 452, 454, and 456 are shaped as arcs having different radii and arc lengths. Electrode 458 is a straight electrode with no curvature. Electrodes 460 and 462 are circular electrodes of different sizes. These electrodes are merely illustrative. The electrodes of medical kit 440 may be electrodes of any suitable radii, arc lengths, lengths, or sizes.

The electrodes of medical kit 440 may be used as either cathodes or anodes either around the wound or in the wound. The specific size and shapes of the electrodes for a wound may be selected as appropriate by a medical professional based on the shape and type of wound. When the electrodes are selected to be placed outside of the wound, the electrodes may be arranged so that there is no space between adjacent electrodes, a small space between adjacent electrodes, or a large space between adjacent electrodes. For example, two electrodes may be selected for placement on opposite sides of the wound. Electrodes 460 and 462 may be used, for example, as cathodes or anodes in the wound in combination with other electrodes outside or surrounding the wound.

Medical kit 440 may also includes one or more control modules 470. In one example, control module 470 may be used as the control module in electrode system 400 shown in FIG. 12. Control module 470 includes ports 472, 474, and 476. Ports 472 and 474 may be configured to selectively receive any of electrodes 450, 452, 454, 456, and 458 and port 476 may be configured to selectively receive electrodes 460 and 462. This configuration prevents electrodes 460 and 462 from being connected to ports 472 and 474 and prevents electrodes 450, 452, 454, 456, and 458 from being connected to ports 476. In addition, the ports and/or electrode connections may be color coded to facilitate connecting the electrodes to the appropriate ports. While control module 470 is illustrated as including three ports, any suitable number of ports may be included.

In accordance with other embodiments of the present invention, an electrode may be incorporated as part of a drainage tube. Drainage tubes are typically flexible and malleable tubes used to drain fluids from parts of a patient's body. For example, drainage tubes can be used to drain fluid from surgical wound cavities. The procedure generally involves placing a flexible hollow tube into a patient's body with the end of the tube located at the site to be drained. The opposite end of the tube can be connected to a wound drainage reservoir, which may, for example, be a vacuum drainage bottle or a pump that periodically helps to drain the fluid.

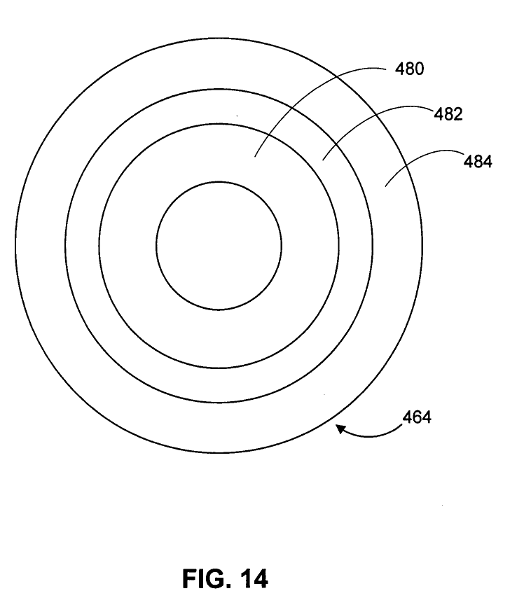

Electrode 464 illustrated in FIG. 13 shows one embodiment of an electrode drainage tube. FIG. 14 is a sectional view of electrode drainage tube 464 of FIG. 13 taken along line 14-14. As shown in FIG. 14, electrode drainage tube 464 may include multiple layers. Inner layer 480 may be made of conventional drainage tube material such as silicone elastomer or PVC. Middle layer 482 is the electrode layer and is made of any suitable conductive material. Outer layer 484 is an electrically conductive adhesive layer that may assist securing the electrode drainage tube to the skin surrounding the opening through which it passes. The middle and outer layers of electrode drainage tube 464 may be present along the entire length of inner layer 480, or may be present along only one or more portions of inner layer 480. In one suitable approach, the middle and outer layers are present along the section of inner layer 480 that is positioned at the opening of the skin during use.

Electrode drainage tube 464 may be used with one or more other electrodes located away from the electrode tube in accordance with the principles of the present invention. Use of electrode drainage tube 464 may help prevent infection of the opening through which the tube passes and may facilitate healing of the opening and any other surrounding damaged tissue, while at the same time provide drainage for any excess fluid.

Electrode drainage tube 464 may be manufactured using a co-extrusion process. For example, the inner, middle, and outer layer of electrode drainage tube 464 may be co-extruded together. In another suitable example, the inner and middle layer of electrode drainage tube 464 may be co-extruded together and outer layer 484 may be applied in a subsequent step. Electrode drainage tube 464 may also be manufactured by applying the middle and outer layers to a conventional drainage tube. For example, this may be done by wrapping and bonding the middle and outer layers about a conventional drainage tube. The middle and outer layers should be sufficiently flexible to allow the electrode drainage tube be positioned as appropriate to drain excess fluids. In some embodiments, the electrode drainage tube may not include the outer layer. The electrode drainage tube may also be manufactured by assembling two or more hollow cylindrical tubes. One of the tubes may include the electrode and another of the tubes may not include an electrode.

The electrodes shown in medical kit 440 and the other electrodes of the present invention, may include a tether or cord of varying length to attach the electrodes to the control module. In other embodiments, the tether or cord may be separate from the electrodes and may be selected as appropriate for coupling electrodes to a control module.

The foregoing is merely illustrative of the principles of this invention and various modifications can be made by those skilled in the art without departing from the scope and spirit of the invention. For example, the electrodes and methods described herein may be used for applications other than wound healing such as scar reductions, wrinkle reductions, improved quality of tissue deposition, hair growth, and on the face and neck after, for example, dermal peeling following laser or chemical facial peels. In addition, the electrode systems and methods may be used in veterinary applications.

What is claimed is:

1. An electrode system, comprising:
a first electrode segment have a first end and a second end, wherein the first electrode segment includes a connection node configured to couple the first electrode segment to a power supply and wherein the first end of the first electrode segment includes a first connection point;
a second electrode segment having a first end and a second end, wherein the first end of the second electrode segment includes a second connection point; and
a first connector configured to electrically couple the first connection point of the first electrode segment to the second connection point of the second electrode segment.

2. The electrode system of claim 1, wherein the second end of the second electrode includes a third connection point, the system further comprising:
a third electrode segment have a first end and a second end, wherein the first end of the third electrode segment includes a fourth connection point; and
a second connector configured to electrically couple the third connection point of the second electrode segment to the fourth connection point of the third electrode.

3. The electrode system of claim 1, wherein the second electrode segment includes a connection node configured to couple the second electrode segment to a power supply.

4. The electrode system of claim 1, wherein the first connector is further configured to physically couple the first end of the first electrode approximately adjacent to the first end of the second electrode.

5. The electrode system of claim 1, wherein the first electrode segment has a first shape and the second electrode segment has a different shape.

6. A method of creating and applying electrodes to a patient, comprising:
selecting a first electrode component of a first shape and size having a first connection point;
selecting a second electrode component of a different shape and size having a second connection point;
using a connector to couple the first connection point of the first electrode component to the second connection point of the second electrode component to create a combined electrode; and
applying the combined electrode to the patient.

7. The method of claim 6, wherein the second electrode component includes a third connection point, the method further comprising:
selecting a third electrode component having a fourth connection point; and
using a second connector to couple the third connection point of the second electrode component to the fourth connection point of the third electrode component.

8. The method of claim 6, further comprising:
selecting the first electrode component and the second electrode component such that the shape of the combined electrode follows the contour of a wound on the patient.

9. The method of 8, further comprising:
selecting a third electrode component; and
applying the third electrode component to the wound.

10. The method of 9, further comprising:
applying a voltage potential across the combined electrode and the third electrode component.

11. A medical electrode kit for use in applying a treatment to a wound or skin, comprising:
a plurality of a first type of electrode segments having a first shape;
a plurality of a second type of electrode segments having a second shape that is different than the first shape; and
a plurality of connectors configured to electrically couple a first electrode segment of the first type of electrode segments and the second type of electrode segments to a second electrode segment of the first type of electrode segments and the second type of electrode segments.

12. The medical electrode kit of claim 11, wherein the plurality of the first type of electrode segments includes electrode segments in two or more sizes and wherein the plurality of the second type of electrode segments includes electrode segments in two or more sizes.

13. The medical electrode kit of claim 11, wherein each electrode segment of the pluralities of the first type of electrode segments and the second type of electrode segments includes at least one connection point, wherein each of the plurality of connectors is configured to electrically couple a connection point of one electrode to a connection point of another electrode.

14. The medical electrode kit of claim 11, further comprising a control module configured to apply a voltage potential across two or more electrode segments.

15. The medical electrode kit of claim 14 wherein each electrode segment of the pluralities of the first type of electrode segments and the second type of electrode segments includes at least one connection node that is configured to couple the electrode segment to the control module.

16. The medical electrode kit of claim 14, wherein the control module is a battery.

17. The medical electrode kit of claim 14, wherein the control module comprises circuitry configured to apply the voltage potential across the two or more electrode segments.

18. The medical electrode kit of claim 14, further comprising at least one of gauze, a scalpel, scissors, tape, and a wound exudates absorber.

19. The medical electrode kit of claim 14, further comprising a diagnostic device.

20. The medical electrode kit of claim 19, wherein the diagnostic device comprises a multi-meter.

21. The medical electrode kit of claim 20, wherein the diagnostic device measures at least one of a voltage and a current of the wound.
